(19) United States
(12) Patent Application Publication
(10) Pub. No.: US 2021/0171377 A1
(43) Pub. Date: Jun. 10, 2021

(54) ONSITE INSTALLATION OR MANUFACTURED PRODUCT OF ECO-FRIENDLY BACTERIAL COMPOSITIONS, METHODS AND SYSTEMS FOR BIOREMEDIATION IN A SHORT DURATION IN DIFFERENT ENVIRONMENTS

(71) Applicant: Mahalakshmi N.A. SRINIVASAN, Mumbai (IN)

(72) Inventor: Mahalakshmi N.A. SRINIVASAN, Mumbai (IN)

(21) Appl. No.: 17/114,173

(22) Filed: Dec. 7, 2020

Related U.S. Application Data
(60) Provisional application No. 62/945,038, filed on Dec. 6, 2019.

(51) Int. Cl. C02F 3/34; C12N 1/20; B09C 1/10

(52) U.S. Cl. CPC C02F 3/341; C02F 2101/32; B09C 1/10; C12N 1/20

(57) ABSTRACT

The present disclosure generally relates to composition(s), method(s) and system(s) for Bioremediation of crude oil spills in a very short duration using eco-friendly bacterial composition(s). This includes bioremediation of crude oil spills in sea, soil, surfaces, lands, water bodies, rhizospheres and ecosystems. The bioremediation of crude oil spills at sea can take place instantaneously. Any bacteria with ability to degrade crude oil, that are eco-friendly and whose growth patterns are well characterized can be used in the composition(s).

Bacteria used in the composition(s) could be New Species, Variants of Known Species and the Known Species.

The technology could be provided as an onsite installation and/or as service, manufactured product at spill sites.

### TECHNICAL FIELD

The present disclosure generally relates to composition(s), method(s) and system(s) for Bioremediation of crude oil spills in a very short duration using eco-friendly bacterial composition(s). This includes bioremediation of crude oil spills in sea, soil, surfaces, lands, water bodies, rhizospheres and ecosystems. The bioremediation of crude oil spills at sea can take place instantaneously. The eco-friendliness refers to the ability to co-exist with different life forms and the numerous benefits rendered to the ecosystem. In particular, the bacteria of the composition(s) which persists in the environment following bioremediation, along with the nutrients present in the surrounding were found to be beneficial for sea microflora and fauna, soil microflora, serves as Plant Growth Promoting Rhizobacteria (PGPR), do not lead to biofilm formation on ship surfaces.

Bacteria could be New Species, Variants of the Known species or the Known species that could be used in the composition(s) with the ability to degrade crude oil, are eco-friendly and whose growth patterns are well characterized can be used in the composition(s).

This known species could be a range of bacteria belonging to the following genus but not restricted only to these:
Alteromonas, Arthrobacter, Bacillus, Brevibacterium, Hydrogenophaga, Lactobacillus, Leuconostoc, Pediococcus, Phyllobacterium, Pseudoalteromonas, Pseudomonas, Streptomyces, Shewanella.

The technology could be provided as an onsite installation and/or as service at oil platform, offshore platform or offshore drilling rig. It could be provided as a service at spill sites, for example at ports or supplied as a manufactured product, for example to the petroleum industries.

### BACKGROUND

Background description includes information that may be useful in understanding the present invention. It is not an admission that any of the information provided herein is prior art to the presently claimed invention, or that any publication specifically or implicitly referenced is prior art.

In this current time, when Microbial Enhanced Oil Recovery (MEOR) is what all are looking for, there is still need for crude oil bioremediation to clean spillage at sea and on soil. The oil spill disasters such as Deepwater Horizon disaster has altered the ocean life and measures are being taken to find a way to clean out crude oil spills, which could have long lasting impact. Reference: The Guardian, Oliver Milman, 28 Jun. 2018, “Deepwater Horizon disaster altered building blocks of ocean life”.

The invention enables bioremediation of crude oil spills, using eco-friendly composition(s); speed of bioremediation depends on the composition(s), method(s) and system(s) being used, the bacteria of the composition(s) which persists in the environment following bioremediation of crude oil spills and along with the nutrients present in the surrounding were found to be beneficial for sea microflora and fauna, soil microflora, serves as Plant Growth Promoting Rhizobacteria (PGPR).

Currently, the sea water environment around the world is becoming toxic to its ecosystem due to oil spills over a period of time, at a number of places around the world. For example, BP oil spill, Exxon Valdez oil spill, and Mumbai oil spill. There have been chemical, mechanical and biological methods available in the art to clean up the oil spills across the globe. However, the results of many of these methods have interfered with the sea and ocean biome. Further, a number of incidences have shown that the present methods treating the oil spills have led to numerous complications in the biome and food web.

Currently, the chemical method includes using one or more dispersants for breaking up oil slicks. Corexit is the most-used dispersant in the Deepwater Horizon oil spill in the Gulf of Mexico, with COREXIT 9527 having been replaced by COREXIT 9500 after the former was deemed too toxic. Moreover, Corexit 9527 and Corexit 9500 dispersants have been banned in USA, UK as it is carcinogenic, mutagenic and highly toxic to sea life.

Further, in one or more situations solidifiers composed of dry hydrophobic polymers that include both adsorb and absorb polymers have been employed which clean up oil spills by changing the physical state of spilled oil from liquid to a semi-solid or a rubber-like material that floats on water. The reaction time for solidification of oil is controlled by the surface area or size of the polymer as well as the viscosity of the oil. However, thoroughness of mixing the solidifiers remains an important issue as reports on early tests conducted revealed that the solidifier reacted with the oil it first got in contact and then formed a harder layer which prevented penetration of any further agents. Consequently, the outer crusts were only solidified with liquid interiors. Further, the effects of solidified oil, partially-solidified oil and raw solidifier on different species of wildlife have not been investigated till date for example, ingestion, contact, adhesion and inhalation of particulate matter.

Mechanical methods including skimmers and booms have been used to remove and contain the spilled oil. Booms are used to contain the oil so that skimmers can remove the oil from the sea water. However, in places where solidifiers have been used, skimmers cannot be put into use. Further, the mechanical methods include the employment of vacuum and centrifuges. In operation, oil is sucked up along with the water, and subsequently a centrifuge is used to separate the oil from the water, thus allowing a tanker to be filled with near pure oil. The water is then returned to the sea along with small amounts of oil to go back as well. However, there are regulations limiting the amount of oil in water that can be returned to the sea, which has hampered the use of centrifuges.

Biological methods include bioremediation by biostimulation, and bioaugmentation approaches at various oil spilled sites. However, one or more changes in bacterial diversity during the field experiment on biostimulation were monitored by denaturing gradient gel electrophoresis (DGGE) analysis of PCR-amplified 16S rDNA fragments. Consequently, the results revealed that the bacterial community were disturbed after the start of treatment, and continued to change from about 45 days to about 60 days and subsequently formed a relatively stable community different from the original community structure. Moreover, sequence analysis on the mmoX gene fragments from the DGGE bands implied that the biostimulation treatment caused a shift of potential dominant sMMO-containing methanotrophs from type I methanotrophs to type II methanotrophs. Further, on conducting bioaugmentation studies it has been observed that the number of exogenous microorganisms decrease shortly after addition to a site.

Fish and shellfish can also digest oil, which could cause changes in reproduction, growth rates or even death. Commercially important species such as oysters, shrimp, grouper, swordfish and tuna also could suffer population declines or become too contaminated to be safely caught and eaten. Depending on just where and when a spill happens, from a few up to hundreds or thousands of birds, fish, mammals, reptiles, corals and other animals and plants can be killed or injured. Gulf of Mexico was a major marine ecosystem threat. Ref: National Oceanic and Atmospheric Administration (NOAA), U.S. Department of Commerce.

Contamination of soil by crude oil could lead to a depression of microbial density and activities even in case of relatively light contamination. Apart from its phytotoxicity, excess oil in soil may also limit the availability of nitrogen (a major plant growth element) in soil. The extent of the effects depends on the original soil properties and the plants exposed to contaminated soil. Ref: Chromic effects of oil spill on soil properties and microflora of a rainforest ecosystem in Nigeria In: Water Air and Soil Pollution 86(1):1-11, 1996.

Crude oil spills could affect the marine microbial communities by reducing the abundance of micro algae and copepods. Ref: Effect of crude oil on marine microbial communities in short term outdoor microcosms. Seung Won JungJoon Sang ParkOh Youn KownJung Hoon KangWon Joon ShimYoung-Ok Kim, The Journal of Microbiology October 2010, Volume 48, Issue 5, pp 594-600

Mangroves are specialized ecosystems found in the intertidal zones of tropical and subtropical regions of the world. Mangroves act as natural buffer zones between land and sea. Mangroves are found along the estuaries, bays and creeks where mud flats are wide and gently sloping. Mangrove plants are specially adopted to withstand high salinity, strong wind and wave actions and oxygen-poor soils. Although the mangroves might appear to be an expanse of draw and weedy plants along the shorelines, these are greatly significant ecosystems and are crucial for the safety and sustenance of our coastal communities and biodiversity alike. They play an important role in the biogeochemical cycles of the coastal environment acting as sources of nutrients to adjacent marine and terrestrial regions. Mangroves help to stabilize climate by moderating temperature, humidity, wind and tides. They actually protect the land from the impact of the sea. In the intertidal areas of creeks and estuaries, mangroves provide critical habitat for a diverse marine and terrestrial flora and fauna. Ref: The Mangroves, By Dr. Maya Mahajan. Soonabai Pirojsha Godrej Marine Ecology Centre, Mumbai. NOAA (National Oceanic and Atmospheric Administration) has its own planning and response considerations for oil spills in the mangrove region. It is essential to protect mangroves from any adverse effects of crude oil spills.

In the absence of a comprehensive solution through chemical, mechanical and biological methods that are available in the art for the cleaning up of oil spills and contributing towards restoration of the ecosystem, there exists a need in the art an innovative way to degrade crude oil pollutants using eco-friendly composition(s) and which could address bioremediation process at a faster rate. The bacteria of the composition(s) which persists in the environment following bioremediation of crude oil spills and along with the nutrients present in the surrounding were found to be beneficial for sea microflora and fauna, soil microflora, serves as Plant Growth Promoting Rhizobacteria (PGPR).

### OBJECTIVES OF THE INVENTION

The objectives of the present disclosure is about bioremediation using eco-friendly bacterial composition(s), method(s) and system(s). In this regard, the aim is to develop eco-friendly bacterial composition(s), method(s), system(s) that enable degradation of crude oil spills in sea, soil, surfaces, lands, water, rhizospheres and ecosystems in a very short duration. Developing composition(s), method(s), system(s) which bioremediates and is beneficial for life forms or co-exists with them, ensures eco-friendliness in the long run. To attain these main objectives, the following objectives had to be met, as follows:

One objective of the invention is to use the eco-friendly bacterial composition(s), method(s) and system(s) for crude oil bioremediation in a very short duration in sea, soil, surfaces, lands, water bodies, rhizospheres and ecosystems.

Another objective is to provide the technology as an onsite installation and/or as service at oil platform, offshore platform or offshore drilling rig. It could be provided as a service at spill sites, for example at ports or supplied as a manufactured product, for example to the petroleum industries.

Another objective of the invention is the use of system(s), method(s) that shall enable the composition(s) to be in direct contact with crude oil spills site, which shall increase the rate at which the bioremediation takes place.

Another objective is to develop eco-friendly bacterial composition(s), method(s), system(s) for bioremediation of crude oil spills such that the bacteria which persists in the environment and along with the nutrients present in the surrounding are found to be beneficial for sea microflora and fauna, soil microflora, serves as Plant Growth Promoting Rhizobacteria (PGPR).

Another objective is to develop eco-friendly bacterial composition(s), method(s), system(s) for bioremediation that could also serve as Plant Growth Promoting Rhizobacteria (PGPR).

Another objective is to develop eco-friendly bacterial composition(s), method(s), system(s) for bioremediation which co-exists with sea microflora.

Another objective is to develop eco-friendly bacterial composition(s), method(s), system(s) for bioremediation which co-exists with soil microflora.

Another objective of the invention is to develop an eco-friendly bacterial composition(s), method(s), system(s) for bioremediation that do not lead to biofilm formation on anti-fouling paint coated ship surfaces.

Various objects, features, aspects and advantages of the present invention will become more apparent from the detailed description of the invention and accompanying figures.

### SUMMARY

The present disclosure generally relates to Bioremediation using eco-friendly bacterial composition(s), method(s) and system(s) for bioremediation of crude oil spills in a short duration. The bacteria of the composition(s) which persists in the environment following bioremediation of crude oil spills and along with the nutrients present in the surrounding were found to be beneficial for sea microflora and fauna, soil microflora, serves as Plant Growth Promoting Rhizobacteria (PGPR), do not lead to biofilm formation on anti-fouling paint coated ship surfaces.

Bacteria could be New Species, Variants of the Known species or the Known species that could be used in the composition(s) with the ability to degrade crude oil, are eco-friendly and whose growth patterns are well characterized can be used in the composition(s).

The known species could could be a range of bacteria belonging to the following genus but not restricted only to these:
Alteromonas, Arthrobacter, Bacillus, Brevibacterium, Hydrogenophaga, Lactobacillus, Leuconostoc, Pediococcus, Phyllobacterium, Pseudoalteromonas, Pseudoarthrobacter, Pseudomonas, Streptomyces, Shewanella.

The bacterial species used in different proportions in the composition(s) were New species belonging to the Pseudomonadaceae family. The whole genome sequences of these bacteria show sequence similarity of (75-85)% to the Pseudomonas species. These bacteria act in synergy and enable crude oil bioremediation in a very short duration in sea, soil, surfaces, lands, water, rhizospheres and ecosystems, apart from being eco-friendly.

Bacteria belonging to the following representative species but the scope of the bacteria in the composition(s) isn't restricted to the following:
Pseudoalteromonas arabiensis, Pseudoalteromonas luteoviolacea, Pseudoalteromonas rubra, Pseudoalteromonas sp., Bacillus amyloliquefaciens, Bacillus firmus, Bacillus flexus, Bacillus lentus, Bacillus pumilus, Bacillus polymyxa, Bacillus megatarium, Bacillus alvei, Bacillus subtilis, Bacillus siamensis, Bacillus sonorensis, Bacillus vallismortis, Bacillus velezensis, Bacillus paralicheniformis, Bacillus vireti, Brevibacterium aureum, Brevibacterium fuscum, Brevibacterium sp., Hydrogenophaga intermedia, Lactobacillus sakei, Lactobacillus rogosae, Lactobacillus plantarum, Lactobacillus pentosus, Lactobacillus paracasei, Lactobacillus panis, Lactobacillus pontis, Lactobacillus reuteri, Lactobacillus fiumenti, Lactobacillus sanfranciscensis, Lactobacillus casei, Lactobacillus coryniformis, Lactobacillus mellis, Lactobacillus brevis, Lactobacillus buchneri, Lactobacillus bulgaricus, Lactobacillus curvatus, Lactobacillus farciminis, Lactobacillus firmentum, Lactobacillus helveticus, Lactobacillis lactis, Lactobacillus reuteri, Lactobacillus johnsonii, Leuconostoc citreum, Leuconostoc pseudomesenteroides, Leuconostoc litchii, Pediococcus acidilactici, Pediococcus damnosus, Pediococcus pentosaceus, Pediococcus cerevisiae, Pediococcus argentinicus, Pediococcus cellicola, Pediococcus claussenii, Pediococcus ethanolidurans, Pediococcus inopinatus, Pediococcus parvenus, Pediococcus pentosaceus, Pediococcus siamensis, Pediococcus stilesii, Phyllobacterium brassicacearum, Phyllobacterium ifriqtyense, Phyllobacterium sophorae, Phyllobacterium catacumbae, Phyllobacterium endophyticum, Phyllobacterium ifriqityense, Phyllobacterium leguminum, Phyllobacterium loti, Phyllobacterium myrsinacearum, Phyllobacterium phragmitis, Phyllobacterium rubiacearum, Phyllobacterium salinisoli, Phyllobacterium sophorae, Phyllobacterium trifolii, Phyllobacterium zundukense, Pseudoalteromonas arabiensis, Pseudoalteromonas luteoviolacea, Pseudoalteromonas rubra, Pseudomonas alkylphenolica, Pseudomonas alcaliphila, Pseudomonas bauzanensis, Pseudomonas clemanceaPseudomonas chlororaphis, Pseudomonas carboxydohydrogena, Pseudomonas glycinae, Pseudomonas koreensis, Pseudomonas kilonensis, Pseudomonas indica, Pseudomonas lini, Pseudomonas rhizosphaerae, Pseudomonas pachastrellae, Pseudomonas grimontii, Pseudomonas graminis, Shewanella sediminis, Streptomyces violaceoruber, Streptomyces ansochromogenes, Streptomyces aureofaciens, Streptomyces noursei, Streptomyces rimosus, Streptomyces sp. S073, Arthrobacter species known to produce therapeutic biomolecules and used based on the fish health condition and fish type. Bacteria could be used individually in a composition or in various permutations and combinations, Pseudarthrobacter chlorophenolicus, Pseudomonas butanovora

The technology could be provided as an onsite installation and/or as service at oil platform, offshore platform or offshore drilling rig. It could be provided as a service at spill sites, for example at ports or supplied as a manufactured product, for example to the petroleum industries.

Various objects, features, aspects and advantages of the inventive subject matter will become more apparent from the following detailed description of preferred embodiments, along with accompanying figures.

### DESCRIPTION OF THE DRAWINGS

The accompanying drawings are included to provide further understanding of the present disclosure, and are incorporated in and constitute a part of this specification. The drawings illustrate exemplary embodiments of the present disclosure and together with the description, serve to explain the principles of the present disclosure.

FIG. 1: illustrates an exemplary photograph depicting the Bioremediation on surface.

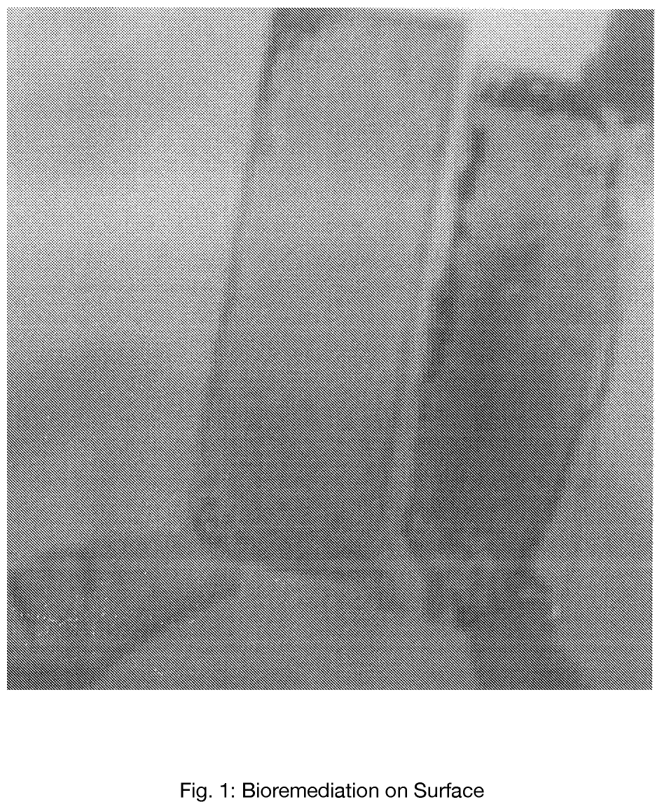

FIG. 2 illustrates an exemplary photograph depicting the Bioremediation in soil and at rhizosphere with the bacteria colonizing the roots of the rhizosphere.

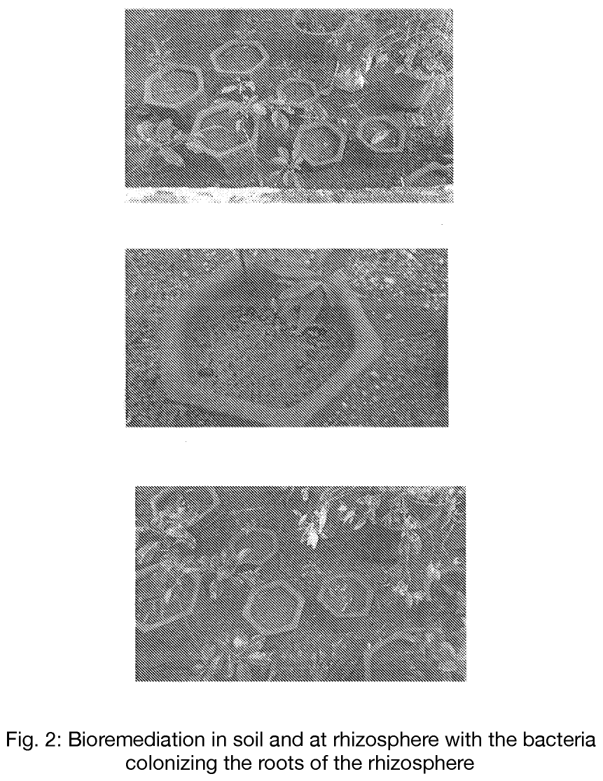

FIG. 3 illustrates an exemplary photograph depicting Non-Hemolytic nature of the bacteria in the composition(s).

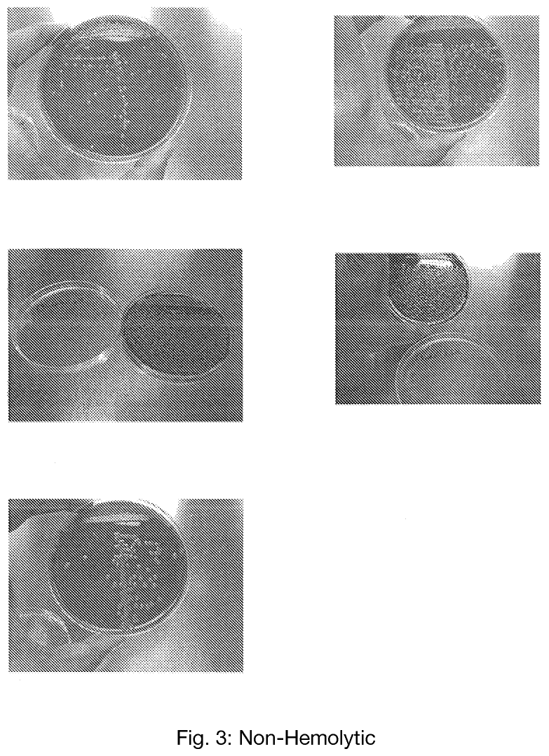

FIG. 4 illustrates an exemplary photograph depicting the instant break-down of crude oil in sea water.

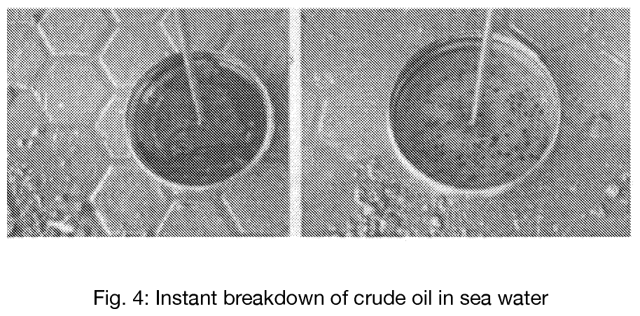

FIG. 5 illustrates an exemplary photograph depicting the ability of the bacteria in the composition(s) to serve as Plant Growth Promoting Rhizobacteria (PGPR).

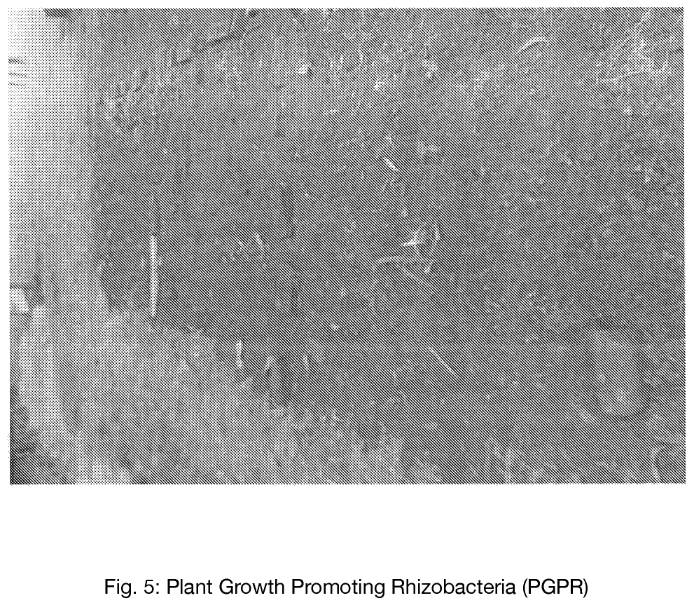

FIG. 6 illustrates an exemplary photograph providing the Schematic representation of the Onsite Installation, Manufacturing of the Product, Mentioned are the Applications.

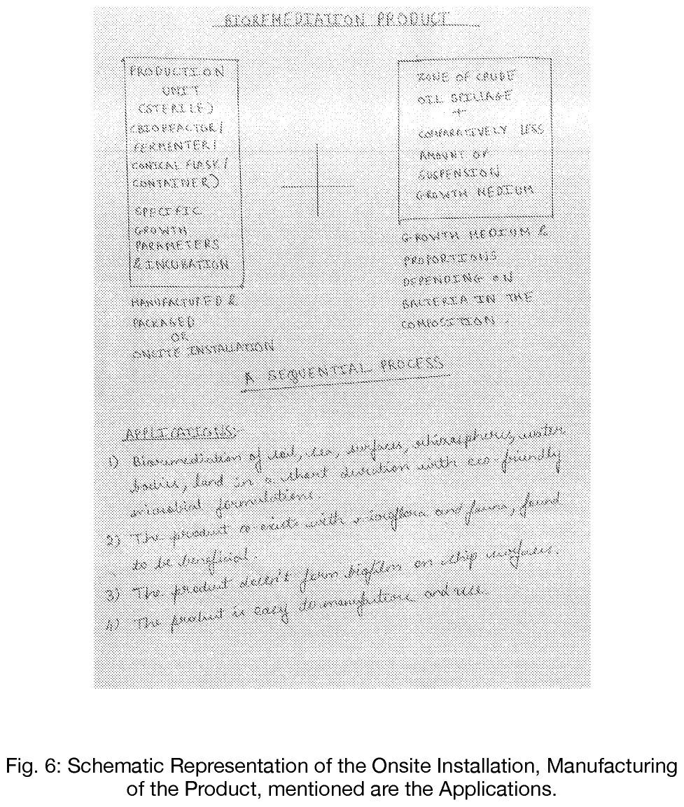

### DETAILED DESCRIPTION:

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents and alternatives falling within the spirit and scope of the present disclosure as defined by the appended claims.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all of the claims.

As used in the description herein and throughout the claims that follow, the meaning of “a”, “an”, “the” includes plural reference unless the context clearly dictates otherwise. Also, as used in the description herein, the meaning of “in” includes “in” and “on” unless the context clearly dictates otherwise.

All method(s) described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Various terms as used herein are shown below. To the extent a term used in a claim is not defined below, it should be given the broadcast definition persons in the pertinent art have given that term as reflected in printed publications and issued patents at the time of filing.

The bacterial isolates whose composition(s) were screened for their eco-friendliness and their ability to bioremediate crude oil spills in a short duration were obtained from Rice Rhizospheres, at Maharashtra, India. 18 bacteria were isolated from these Rice rhizospheres. Each of them were tested for both the criterion of eco-friendliness and bioremediation of crude oil spill in a very short duration. The eco-friendliness of these bacterial composition(s) were screened based on the following criterions: the bacteria of the composition(s) which persists in the environment following bioremediation of crude oil spills and along with the nutrients present in the surrounding were found to be beneficial for sea microflora and fauna, soil microflora, serves as Plant Growth Promoting Rhizobacteria (PGPR), are non-pathogenic, don't lead to biofilm formation of ship surfaces such as hull coated with anti-fouling paint. In this process of screening for bacteria whose composition(s) are eco-friendly based on above mentioned criterions and ability to bioremediate crude oil spills in a short duration, bacteria were shortlisted which fulfilled these criterions. The studies involved usage of various growth medium such as Nutrient Broth, Zobell Marine Broth, Artificial Sea Media, Artificial Soil Media. Bushnell and Haas wasn't the preferred medium for this study, as the aim was to study bioremediation abilities in the presence of other carbon sources. The studies were further done in soil surrounding the mangroves. Random sampling of sea water and soil was done to obtain sea microflora isolates and soil microflora isolates. These were used to study their interactions with the bacteria which were part of the composition(s). The check for biofilm formation on ship surfaces involved observations and interpretations using ESEM. These bacteria in synergy, in the composition(s) could breakdown crude oil in a very short duration and almost instantaneously, provided the methodology of preparing and using the composition(s) were followed. These results were observed in the presence of other carbon sources. Further, each of these bacteria (individually and in combination) which were found highly beneficial for ecosystem and biodegraded crude oil spills in a short duration in synergy are New Species belonging to the family Pseudomonadaceae.

Bacteria could be New Species, Variants of the Known species or the Known species that could be used in the composition(s) with the ability to degrade crude oil, are eco-friendly and whose growth patterns are well characterized can be used in the composition(s).

The known species could could be a range of bacteria belonging to the following genus but not restricted only to these:
Alteromonas, Arthrobacter, Bacillus, Brevibacterium, Hydrogenophaga, Lactobacillus, Leuconostoc, Pediococcus, Phyllobacterium, Pseudoalteromonas, Pseudoarthrobacter, Pseudomonas, Streptomyces, Shewanella.

Bacteria belonging to the following representative species but the scope of the bacteria in the composition(s) isn't restricted to the following:
Pseudoalteromonas arabiensis, Pseudoalteromonas luteoviolacea, Pseudoalteromonas rubra, Pseudoalteromonas sp., Bacillus amyloliquefaciens, Bacillus firmus, Bacillus flexus, Bacillus lentus, Bacillus pumilus, Bacillus polymyxa, Bacillus megatarium, Bacillus alvei, Bacillus subtilis, Bacillus siamensis, Bacillus sonorensis, Bacillus vallismortis, Bacillus velezensis, Bacillus paralicheniformis, Bacillus vireti, Brevibacterium aureum, Brevibacterium fuscum, Brevibacterium sp., Hydrogenophaga intermedia, Lactobacillus sakei, Lactobacillus rogosae, Lactobacillus plantarum, Lactobacillus pentosus, Lactobacillus paracasei, Lactobacillus panis, Lactobacillus pontis, Lactobacillus reuteri, Lactobacillus frumenti, Lactobacillus sanfranciscensis, Lactobacillus casei, Lactobacillus coryniformis, Lactobacillus mellis, Lactobacillus brevis, Lactobacillus buchneri, Lactobacillus bulgaricus, Lactobacillus curvatus, Lactobacillus farciminis, Lactobacillus firmentum, Lactobacillus helveticus, Lactobacillis lactis, Lactobacillus reuteri, Lactobacillus johnsonii, Leuconostoc citreum, Leuconostoc pseudomesenteroides, Leuconostoc litchii, Pediococcus acidilactici, Pediococcus damnosus, Pediococcus pentosaceus, Pediococcus cerevisiae, Pediococcus argentinicus, Pediococcus cellicola, Pediococcus claussenii, Pediococcus ethanolidurans, Pediococcus inopinatus, Pediococcus parvenus, Pediococcus pentosaceus, Pediococcus siamensis, Pediococcus stilesii, Phyllobacterium brassicacearum, Phyllobacterium ifriqtyense, Phyllobacterium sophorae, Phyllobacterium catacumbae, Phyllobacterium endophyticum, Phyllobacterium ifriqiyense, Phyllobacterium leguminum, Phyllobacterium loti, Phyllobacterium myrsinacearum, Phyllobacterium phragmitis, Phyllobacterium rubiacearum, Phyllobacterium salinisoli, Phyllobacterium sophorae, Phyllobacterium trifolii, Phyllobacterium zundukense, Pseudoalteromonas arabiensis, Pseudoalteromonas luteoviolacea, Pseudoalteromonas rubra, Pseudomonas alkylphenolica, Pseudomonas alcaliphila, Pseudomonas bauzanensis, Pseudomonas clemanceaPseudomonas chlororaphis, Pseudomonas carboxydohydrogena, Pseudomonas glycinae, Pseudomonas koreensis, Pseudomonas kilonensis, Pseudomonas indica, Pseudomonas lini, Pseudomonas rhizosphaerae, Pseudomonas pachastrellae, Pseudomonas grimontii, Pseudomonas graminis, Shewanella sediminis, Streptomyces violaceoruber, Streptomyces ansochromogenes, Streptomyces aureofaciens, Streptomyces noursei, Streptomyces rimosus, Streptomyces sp. S073, Arthrobacter species known to produce therapeutic biomolecules and used based on the fish health condition and fish type. Bacteria could be used individually in a composition or in various permutations and combinations, Pseudarthrobacter chlorophenolicus, Pseudomonas butanovora

Bioremediation of crude oil spills in a short duration with eco-friendly bacterial composition(s) acting in synergy, method(s), system(s) in sea, soil, surfaces, lands, water, rhizospheres and ecosystems: The field trial studies were done. The various criterions to attain degradation of crude oil spills depended on the medium being used, the inoculum density, incubation time, whether the composition(s) was used in a liquid form or used as a cell mass, along with other components of the composition(s), usage of system(s) to enable direct contact of the composition(s) with crude oil to be biodegraded. The bioremediation of crude oil spills happened anytime from almost instantaneously to around 144 hours, and were dependent on the criterions mentioned above.

Composition(s) for bioremediation of crude oil spills: The composition(s) that enabled bioremediation of crude oil spills comprise of bacteria that are New Species belonging to the family Pseudomonadaceae in proportions ranging from (0.5: 0.5:2) to (1:1:3) plus culture media such as Nutrient Broth or customized culture medium. Various nutrients could be used in the composition(s) depending on the medium preferences of the bacteria in the composition(s). The amount of culture media is proportional to the amount of bacterial suspension being used which in turn depends on the area or volume being bioremediated. A minimal of 48 hour old incubated bacterial cultures were used in the composition(s) for bioremediation of crude oil spills in a very short duration. For instant bioremediation of crude oil spills in sea water, bacterial suspension cultures incubated for a minimum of (3-7 days) comprising of bacteria were preferred over 48 hour old incubated bacterial suspension cultures. The composition(s) could comprise of bacterial suspensions incubated for more than 7 days. Bacterial cell mass could be used instead of bacterial suspensions. The bacteria act in synergy to breakdown the crude oil. In case bacterial cell mass based composition(s) would be used to biodegrade crude oil spills, then culture media such as Nutrient Broth or any customized culture medium could be added, just enough to enable the mixing of cell mass of the composition(s) and crude oil in the system(s) as mentioned above.

The bacteria of the composition(s) which persists in the environment following bioremediation of crude oil spills and along with the nutrients present in the surrounding were found to be beneficial for sea microflora and fauna, soil microflora, serves as Plant Growth Promoting Rhizobacteria (PGPR).

The composition(s) for bioremediation of crude oil spills has bacteria which could serve as Plant Growth Promoting Rhizobacteria (PGPR).

The process involved in preparing the composition(s) is adding the culture media to the environment being bioremediated and then adding sequentially the bacterial suspensions to the environment to be bioremediated. Bacterial suspensions incubated for a minimum time of 48 hours is preferred and a minimum incubation of 3-7 days or more is preferred, for instant breakdown of crude oil at sea water. The composition(s) prepared in a prescribed manner along with system(s) enable the instant breakdown of crude oil spills at sea water. The bioremediation could be achieved at sea, soil, surfaces, lands, water, rhizospheres and ecosystems.

To achieve direct interaction of the composition(s) with spilled crude oil, in water, custom designed equipment which will mix the composition(s) and crude oil, with minimal involvement of water such as sea water is needed. This will hasten the process of biodegradation of crude oil. Custom designed equipment could be designed to contain maximum of crude oil spillage at the surface of sea water with minimal sea water within this container. Following this, the culture media which is part of the composition(s) is added. This is followed by sequential addition of the bacterial suspension cultures incubated for about (3-7 days) minimum or more, to enable instant breakdown of crude oil spills in the sea water. Once this process is completed, the composition(s) is mixed with the crude oil and sea water within the container, to enable complete degradation. The mixing could be done for a time frame of 24 hours to ensure complete bioremediation.

The bacterial composition(s) were found to bioremediate crude oil spills in soil surrounding the mangroves and colonised the roots of mangroves.

The bacterial composition(s) for bioremediation were found to coexist with sea microflora and soil microflora. These isolates of sea and soil were obtained by random sampling and enrichment of sea sample and soil sample.

Each of the bacterial composition(s) did not lead to biofilm formation. The bacteria used in the composition(s) were incubated for a minimal time of 48 hours before being used in the composition(s). The studies were done using carbon steel which were treated each of the composition(s) and ESEM studies carried out. The carbon steel used were of two types—antifouling paint coated steel and only steel (not coated with anti-fouling paint). These studies indicated that the bacteria do not lead to biofilm formation on ship surfaces. ESEM studies indicated that anti-fouling paint coated steel had much lesser bacteria seen in comparison to the steel which wasn't coated with anti-fouling paint. However, in both cases, biofilm formation wasn't observed. For this study, each of the steel was immersed into the bacterial composition(s) (suspension), incubation for at least a week and then ESEM analysis done.

The bacterial composition(s) did not lead to hemolysis. The studies were carried out using the Blood Agar plates.

The bacterial composition(s) were sensitive to a range of Antibiotics. The Antibiotic Sensitivity Testing was done using Mueller Hinton agar plates with a range of antibiotics. The antibiotics used for the study were Amoxyclav (30 mcg), Ampicillin (10 mcg), Aztreonam (30 mcg), Ceftazidime (30 mcg), Clindamycin, Co-Trimoxazole (25 mcg), Furozolidone (50 mcg), Metronidazole (4 mcg), Nitrofurazone (100 mcg), Penicillin-G (10 units), Ticarcillin (75/10 mcg).

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### EXAMPLES

### Example 1

Bioremediation of crude oil in synergy by bacteria with studied using various culture media:

While one isolates microbes from zones where crude oil spills or petroleum product spills have happened for using in bioremediation, here the aim was to find bacteria with 2 main attributes of being able to bioremediate crude oil spills in a short duration and being eco-friendly. Hence rhizospheres were chosen for isolating these microbes. The eco-friendliness was determined by the ability of these isolates to co-exist with other life forms or be beneficial to other life forms. They were also screened to see if they lead to biofilm formation on ship surfaces. With these criterion, New species of bacteria belonging to family Pseudomonadaceae were isolated that could in synergy bioremediate crude oil spills in a short duration and were found to be eco-friendly. The various growth medium used for the ability to bioremediate crude oil spills includes Nutrient agar plates, Zobell Marine agar, Artificial soil agar plate, Bushnell and Haas media. Accordingly, medium having carbon sources other than crude oil were used while developing a composition(s) which can bioremediate crude oil spills in a very short duration. This is a more realistic situation in comparison to Bushnell and Haas as in real environments there are carbon sources other than crude oil. The bioremediation in synergy was observed in the different growth medium, however the incubation time taken for the bioremediation differed based on the medium being used. For these studies, crude oil was added to the growth media plate, and cell pellets of these bacteria used in various proportions. The composition(s) was optimised to get the bioremediation of crude oil spills done in synergy in a short duration. Accordingly, with this experiment we found a composition(s) of bacterial isolates which could degrade crude oil spills in synergy and in presence of other carbon sources as well. The bioremediation took from 96 hours to a week.

### Example 2

Bioremediation of crude oil in synergy by bacteria with in Nutrient broth; in other words in presence of other carbon sources:

In this experiment, 16 drops of crude oil (1500 ppm) and cell pellets in the proportions were added to 400 ml of sterile Nutrient Broth medium. One example is using 0.84 gm, 0.87 gm, 1.08 gm of bacteria (New Species belonging to family Pseudomonadaceae) in 400 ml sterile Nutrient Broth, containing 16 drops of heavy crude oil. The degradation was observed in 96 hours. In this example, like the above example, it was the cell pellet being used, as obtained from the suspension culture. Further, this cell pellet was used in a 400 ml medium. No oil coating the glass surface was seen from third day of incubation. The complete degradation was taking time and on 4th day when 10 ml of 50 hour old incubated suspension culture of one of these bacteria was added, immediate breakdown of crude oil was observed.

### Example 3

Bioremediation of crude oil spill in synergy by cell mass of bacteria (New Species belonging to family Pseudomonadaceae) on a slide with crude oil. This concept could be used for breaking down crude oil in a very short duration:

In this experiment colonies of bacteria (New Species belonging to family Pseudomonadaceae) were incubated for a minimum time of 48 hours were chosen. The proportions of cell mass used were in the range of (0.5:0.5:2) to (1:1:3). On a slide, a drop of crude oil was placed and colonies of each of these bacteria acting in synergy, were introduced sequentially. There was constant mixing done while these colonies were being introduced to the slide. In this case, breakdown of crude oil was observed in 5-20 minutes. Unlike the above two examples, in this experiment the composition(s) was in direct contact of crude oil. The direct contact of composition(s) with crude oil at sea water could be achieved using system(s).

### Example 4

Instant bioremediation of crude oil spills in sea water has been described as follows:

To attain this, the following proportions were used for 100 ml of sea water, to which 50 drops of crude oil were added: ¼ volume of sea water is the culture media (Nutrient Broth was used in this case). Then, (3-7) days minimum or more incubated bacterial suspensions of bacteria (New Species belonging to family Pseudomonadaceae) in proportions ranging from (0.5:0.5:2) to (1:1:3) were added sequentially with constant mixing. The above experiment was setup in an equipment which could contain more of the crude oil spills (at the surface of the sea) and minimal of sea water. The mixing happened in this custom designed equipment. Following this, there is instant breakdown of crude oil spill but the mixing procedure continues to ensure complete disintegration and breakdown. The mixing could continue for about 24 hours, to ensure the process is completed. Then the custom designed system(s) could be shifted to the next location in the sea to bioremediate crude oil spills.

### Example 5

Bioremediation of crude oil spill in synergy by bacteria in soil surrounding mangroves:

The bioremediation composition(s) was added to the soil surrounding the mangroves; one pot with crude oil and one without crude oil in it. The composition(s) used were New Species of bacteria belonging to family Pseudomonadaceae in proportions ranging from (0.5:0.5:2) to (1:1:3) plus culture media, Nutrient broth was used in this case. The crude oil was bioremediated within 1 week time. The bacteria in the composition(s) were found to colonise the roots of the mangroves. These bacteria have been found to serve as Plant Growth Promoting Rhizobacteria (PGPR) as observed in soil devoid of fertilisers.

### Example 6

The bacteria (New Species belonging to family Pseudomonadaceae) co-existed with microbial isolates obtained from sea sample and soil sample:

The sea and soil samples were randomly sampled and enriched to obtain the sea isolates and soil isolates. Fungi and bacteria were isolated and preserved on Nutrient agar slants. These isolates were then studied for their co-existence with the bacteria of the composition(s). The interactions of these isolates, each of the isolates from sea sample and soil sample were studied with bacteria in the composition(s). This led to the interpretation that the bacteria of the composition(s), independently or in combination co-exist with sea microflora and soil microflora.

### Example 7

The composition(s) with bacteria (New Species belonging to the family Pseudomonadaceae) in various proportions ranging from (0.5:0.5:2) to (1:1:3) plus culture media (Nutrient Broth used in this case) did not lead to biofilm formation on ship surfaces, as interpreted based on ESEM studies:

The study involved analyzing carbon steel coupons which were placed in Nutrient broth with the composition(s) and crude oil for a week. The coupons were then observed under ESEM for biofilm formation. In the case of carbon steel coupons coated with anti fouling paint, no bacteria were observed. In the case of carbon steel coupons without the antifouling paint, a few bacterial cells were observed in ESEM studies, but no biofilm formation.

### Example 8

The composition(s) comprising of bacteria (New Species Belonging to the family Pseudomonadaceae) in various proportions ranging from (0.5:0.5:2) to (1:1:3) plus culture media (Nutrient Broth used in this case) did not lead to blood hemolysis:

The studies were carried out using Blood Agar plates. Bacterial growth was observed but no hemolysis observed.

### Example 9

The composition(s) comprising of bacteria (New Species belonging to family Pseudomonadaceae) in various proportions ranging from (0.5:0.5:2) to (1:1:3) plus culture media (Nutrient Broth used in this case) were studied for their sensitivity to a range of antibiotics:

A range of antibiotics were chosen for Antibiotic Sensitivity Testing—AST studies of the bacteria part in the composition(s). They were: Amoxyclav (30 mcg), Ampicillin (10 mcg), Aztreonam (30 mcg), Ceftazidime (30 mcg), Clindamycin, Co-Tri-moxazole (25 mcg), Furozolidone (50 mcg), Metronidazole (4 mcg), Nitrofurazone (100 mcg), Penicillin-G (10 units), Ticarcillin (75/10 mcg).

### Example 10

The technology could be provided as an onsite installation and/or as service at oil platform, offshore platform or offshore drilling rig. It could be provided as a service at spill sites, for example at ports or supplied as a manufactured product, for example to the petroleum industries. The schematic represents depicts each of these mode of commercialization.

### Advantages of the Invention

The present disclosure provides an innovative solution to Bioremediate using eco-friendly bacterial composition(s), unlike the existing technologies which only focus on degradation of crude oil spills and not the implications of using the technologies on the ecosystem, in the long run.

The present disclosure provides an innovative way to bioremediate crude oil spills at sea, soil, surfaces, lands, water, rhizospheres and ecosystems in short durations.

The present disclosure provides an innovative way to bioremediate crude oil spills almost instantaneously in sea water.

The present disclosure provides solution to enable bioremediation of crude oil spills in soil surrounding mangroves.

The present disclosure provides solution to bioremediate crude oil spills with eco-friendly bacterial composition(s) without forming biofilms on ship surfaces coated with anti-fouling paints. The eco-friendliness is that the bacteria of the composition(s) which persists in the environment following bioremediation of crude oil spills and along with the nutrients present in the surrounding were found to be beneficial for sea microflora and fauna, soil microflora, serves as Plant Growth Promoting Rhizobacteria (PGPR).

The present disclosure provides composition(s) which don't lead to hemolysis and has sensitivity to a range of antibiotics.

The technology could be provided as an onsite installation and/or as service at oil platform, offshore platform or offshore drilling rig. It could be provided as a service at spill sites, for example at ports or supplied as a manufactured product, for example to the petroleum industries.

1. Onsite production or supply as Manufactured product of the composition(s) to the Bioremediation site comprising of:
Bacteria that could be New Species,
Bacteria that could be variant of the known species,
Bacteria that are known species belonging to a range of genera,
Bacteria that are known species and belong to the following representative genus:
Alteromonas, Arthrobacter, Bacillus, Brevibacterium, Hydrogenophaga, Lactobacillus, Leuconostoc, Pediococcus, Phyllobacterium, Pseudoalteromonas, Pseudomonas, Streptomyces, Shewanella.
Bacteria belonging to the representative species:
Pseudoalteromonas arabiensis, Pseudoalteromonas luteoviolacea, Pseudoalteromonas rubra, Pseudoalteromonas sp., Bacillus amyloliquefaciens, Bacillus firmus, Bacillus flexus, Bacillus lentus, Bacillus pumilus, Bacillus polymyxa, Bacillus megatarium, Bacillus alvei, Bacillus subtilis, Bacillus siamensis, Bacillus sonorensis, Bacillus vallismortis, Bacillus velezensis, Bacillus paralicheniformis, Bacillus vireti, Brevibacterium aureum, Brevibacterium fuscum, Brevibacterium sp., Hydrogenophaga intermedia, Lactobacillus sakei, Lactobacillus rogosae, Lactobacillus plantarum, Lactobacillus pentosus, Lactobacillus paracasei, Lactobacillus panis, Lactobacillus pontis, Lactobacillus reuteri, Lactobacillus frumenti, Lactobacillus sanfranciscensis, Lactobacillus casei, Lactobacillus coryniformis, Lactobacillus mellis, Lactobacillus brevis, Lactobacillus buchneri, Lactobacillus bulgaricus, Lactobacillus curvatus, Lactobacillus farciminis, Lactobacillus fermentum, Lactobacillus helveticus, Lactobacillis lactis, Lactobacillus reuteri, Lactobacillus johnsonii, Leuconostoc citreum, Leuconostoc pseudomesenteroides, Leuconostoc litchii, Pediococcus acidilactici, Pediococcus damnosus , Pediococcus pentosaceus, Pediococcus cerevisiae, Pediococcus argentinicus, Pediococcus cellicola, Pediococcus claussenii, Pediococcus ethanolidurans, Pediococcus inopinatus, Pediococcus parvenus, Pediococcus pentosaceus, Pediococcus siamensis, Pediococcus Phyllobacterium brassicacearum, Phyllobacterium ifriqiyense, Phyllobacterium sophorae, Phyllobacterium catacumbae, Phyllobacterium endophyticum, Phyllobacterium ifriqiyense, Phyllobacterium leguminum, Phyllobacterium loti, Phyllobacterium myrsinacearum, Phyllobacterium phragmitis, Phyllobacterium rubiacearum, Phyllobacterium salinisoli, Phyllobacterium sophorae, Phyllobacterium trifolii, Phyllobacterium zundukense, Pseudoalteromonas arabiensis, Pseudoalteromonas luteoviolacea, Pseudoalteromonas rubra, Pseudomonas alkylphenolica, Pseudomonas alcaliphila, Pseudomonas bauzananensis, Pseudomonas clemanceaPseudomonas chlororaphis, Pseudomonas carboxydohydrogena, Pseudomonas glycinae, Pseudomonas koreensis, Pseudomonas kilonensis, Pseudomonas indica, Pseudomonas lini, Pseudomonas rhizosphaerae, Pseudomonas pachastrellae, Pseudomonas grimontii, Pseudomonas graminis, Shewanella sediminis, Streptomyces violaceoruber, Streptomyces ansochromogenes, Streptomyces aureofaciens, Streptomyces noursei, Streptomyces rimosus, Streptomyces sp. S073, Arthrobacter species known to produce therapeutic biomolecules and used based on the fish health condition and fish type. Bacteria could be used individually in a composition or in various permutations and combinations, Pseudarthrobacter chlorophenolicus, Pseudomonas butanovora
Growth medium which could be simple, complex, selective or customized,
A proportionally small amount of the suspension growth medium that is added to the spill site followed by the addition of cultivated bacteria,
Sequential process involving constant mixing,
Variations to this basic rule in the method of preparation depending on where it is being used,
Variations in the composition(s) and their proportions in permutations and combinations and along with the system(s) and method(s) renders the various benefits characteristic of the composition(s).

2. The composition as recited in claim 1, wherein the incubation time depends on the bacteria, growth medium used, preference for the growth medium studied using growth patterns and inoculum density. Generally, a minimum incubation of 48 hours is preferred
and is dependent on the growth conditions of the bacteria used in the composition(s).

3. The composition as recited in claim 1, wherein the shelf life of the composition(s) depends on the bacteria, growth medium used in preparation of the composition(s) and the best to use time could range from a month to six months from the date of manufacturing.

4. The composition as recited in claim 1, wherein the bacteria could be a probiotic bacteria.

5. The composition as recited in claim 1, wherein the bacteria could belong to a single species or could be combination of multiple species.

6. The composition of claim 1, where in the composition(s) could be fine tuned for the spill site.

7. The composition(s) as recited in claim 1, the method(s) and system(s) characterized by:
Ability to bioremediate crude oil spills in sea, soil, surfaces, lands, water bodies, rhizospheres and ecosystems in short durations ranging from instantaneous to a week, Ability to bioremediate crude oil spills in sea, soil, surfaces, lands, water bodies, rhizospheres and ecosystems in the presence of other carbon sources,
Ability to bioremediate crude oil spillage in sea water almost instantaneously to about within a day,
Usage of composition(s) which are eco-friendly, the bacteria that persists in the environment along with surrounding nutrients following bioremediation of crude oil spills have been found to co-exist with sea microflora and fauna, soil microflora and fauna, serve as Plant Growth Promoting Rhizobacteria (PGPR),
Non-hemolytic composition(s) and sensitivity to a range of antibiotics, Using bacteria with well characterized growth patterns and which are eco-friendly.

8. The composition(s), method(s), system(s) for bioremediation of crude oil spills; their rates of bioremediation characterized by:
The area or volume of the zone being bioremediated,
The ppm of crude oil in the environment,
The amount of composition(s) to be used,
The growth incubation time for bacteria used in the composition(s),
The amount of culture media to be used in the composition(s) for bioremediation,
The method(s) used in preparing the composition(s), in terms of the incubation times for the bacteria, the form in which the bacteria is being used—suspension culture or cell mass, the proportions of bacteria being used and acting in synergy, the kind and amount of growth media being used in the composition(s),
The system(s) being used to hasten the bioremediation process.

9. The composition(s) for bioremediation of crude oil spills of claim 8, further comprising comprises of the bacteria, the media which hastens the bioremediation process and not just serves as a growth medium for the bacteria.

10. The composition(s) of claim 1, wherein the media could be any nutritive media or growth media, defined or customized in formulation(s). One example is Nutrient Broth.

11. The composition(s) of claim 1, wherein bacterial combination in the composition(s) act in synergy to bring about bioremediation of crude oil spills.

12. The composition(s) of claim 1, wherein the bacteria could be used individually in the composition(s) or in different proportions and in various permutation and combinations, at sea, soil, surfaces, lands, water bodies, rhizospheres and ecosystems to attain bioremediation synergistically.

13. The composition(s) of claim 1, wherein bacteria with ability to degrade crude oil, that are eco-friendly and whose growth patterns are well characterized could be used for the bioremediation of crude oil spills.

14. A method for preparing the composition(s) of claim 1, in terms of the incubation times for the bacteria, the form in which the bacteria are being used, as suspension culture or cell mass, the proportions of bacteria being used and acting in synergy or if species of one type of bacteria is used in the composition(s), the kind and amount of media being used in the composition(s), the sequence of addition of the composition(s) to the environment to be bioremediated.

15. The method of bioremediation of claim 8, wherein the media, in suspension or dehydrated form is added to the spill site, followed by the addition of the incubated bacteria, involving constant mixing; the proportions of the components, the ingredients of the components, methodologies of sequential addition could vary.

16. The method(s) of bioremediation of crude oil spills in sea water in the presence of other carbon sources, wherein the method enables instant breakdown of crude oil spills, the method comprising:
Using a system to contain the crude oil at the surface of sea with minimal amount of sea water within this system,
Using the composition in a particular method to enable instant bioremediation in sea water, the proportions could be:
100 ml sea water to which 50 drops of crude oil has been added. To this 100 ml sea water with 50 drops of crude oil, (10-50) ml of growth media is added, for example Nutrient Broth is added. Then, the bacterial suspensions are added in the proportions ranging from (0.5:0.5:2) to (1:1:3) in various permutations and combinations for (New species belonging to family Pseudomonadaceae and showing 75%-85% similarity to genus Pseudomonas was isolated and used in the composition). The bacterial cultures were introduced sequentially, either as suspension cultures or as cell mass. This formulation is then mixed well. The custom designed system(s) enables mixing to ensure complete bioremediation of crude oil spills in sea water.
The bioremediated water is passed through the biofiltration system before entering the sea water.

17. The method(s) of claim 14, wherein bioremediation of crude oil spills (1500 ppm-16 drops of heave crude oil) happened within 48 hour as observed in 400 ml sterile Nutrient Broth. The complete bioremediation was achieved on further adding 10 ml of 50 hour old bacterial cultures.

18. The method of claim 14, wherein the bioremediation on surfaces could be achieved instantaneously.

19. The method(s) of bioremediation of claim 8, wherein the methods are used for crude oil spills in soil, soil surrounding rhizospheres, in the presence of other carbon sources, wherein the method(s) comprises of:
Adding the formulation(s) incubated for a minimum of 48 hours along with growth media such as Nutrient Broth to the soil, soil surrounding the rhizospheres. The proportion of the bacteria (New species belonging to family Pseudomonadaceae and showing 75%-85% similarity to genus Pseudomonas was isolated and used in the composition) in the composition(s) range from (0.5:0.5:2) to (1:1:3) in various permutations and combinations.

20. The method(s) of bioremediation of claim 8, wherein the methods are used for crude oil spills on surfaces using bacterial cell formulation(s) cell mass or bacterial suspension culture(s) in proportions ranging from (0.5:0.5:2) to (1:1:3) in various permutations and combinations.

21. (canceled)
